(19) United States
(12) Patent Application Publication
(10) Pub. No.: US 2001/0003797 A1
(43) Pub. Date: Jun. 14, 2001

(54) Degradable disposable diaper

(76) Inventors: Cesar Montemayor Guevara, Garcia (MX); Oscar J. Kat, Garcia (MX); Carlos E. Richer, Garcia (MX); Brian E. Cermak, Spring, TX (US); Joseph G. Gho, Vancouver (CA); David M. Wiles, Victoria (CA)

Correspondence Address: David A. Rose, CONLEY, ROSE & TAYON, P.C., P.O. Box 3267, Houston, TX, 77253-3267, US

(21) Appl. No.: 09/730,050

(22) Filed: Dec. 5, 2000

Related U.S. Application Data
(63) Continuation-in-part of application No. 09/658,921, filed on Sep. 11, 2000.

(30) Foreign Application Priority Data
Dec. 6, 1999 (MX) 48508

(51) Int. Cl. A61F 13/15; A61F 13/20

(52) U.S. Cl. 604/364; 604/374; 604/366; 604/367

(57) ABSTRACT

A degradable disposable diaper includes one or more sheets made of polyolefin with the polyolefin including a prodegradant causing the sheet to degrade. The prodegradant includes a metal compound such as a metal selected from the group consisting of cobalt, cerium, and iron. The preferred metal compound is a metal carboxylate. The polyolefin is preferably polyethylene or polypropylene. A secondary polyolefin may be used to aid the incorporation of the prodegradant into the primary polyolefin. A filler may also be used with the polyolefin and prodegradant. The filler preferably has a particle size less than 150 mesh and is free of water. The filler is preferably calcium carbonate having a 1 to 10 micron particle size. The sheet contains between about 0.001 and about 15 weight % prodegradant and most preferably between about 0.01 and about 3 weight % prodegradant. The sheet also includes up to about 15 weight % filler. The diaper also includes a degradable absorbent core.

### CROSS-REFERENCE TO RELATED APPLICATION

This is a continuation-in-part application of pending U.S. patent application Ser. No. 09/658,921, filed Sep. 11, 2000, which claims the benefit of Mexico Application No. 48508, filed Dec. 6, 1999, both hereby incorporated herein by reference.

### TECHNICAL FIELD

The present invention relates to an improved disposable diaper and more specifically to a disposable diaper that is degradable and environmentally safe.

### BACKGROUND OF THE INVENTION

The first diapers used were cloth diapers, normally made of cotton. These diapers had to be cleaned, washed and pressed for reuse. This task was both inefficient and unpleasant.

In the early 60's disposable diapers made of an absorbent material were introduced into the market place. These disposable diapers alleviated some of the problems associated with cloth diapers, but required the use of waterproof drawers to prevent moisture from soaking through to the outside. During the 70's improvements in disposable diapers revolutionized diapering. Such disposable diapers generally consisted of an absorbent pad, a liquid permeable topsheet covering the pad and a liquid impervious backsheet for containing the liquid waste within the absorbent pad.

In the last few years disposable diapers have been further improved. These improvements were focused on increasing the absorption rate of the pad and water retention properties. At the same time, there was an improvement in the diaper design to make the diaper more comfortable and to avoid leakage.

In eliminating many of the problems associated with earlier diapers, however, the improved disposable diapers have created new problems. In particular such diapers have created the environmentally unsafe practice of disposing the plastic backsheet material in landfills. Such plastic materials are not easy to degrade, in fact their biodegradation could require decades. This represents a serious environmental problem due to the ever increasing use of landfills for containing today's ever growing volume of garbage.

Owing to the nature of today's disposable diapers, many adults dispose soiled diapers by merely tossing them in the household garbage. The diapers are then transported to the municipal landfill and disposed of without any treatment whatsoever. Thus, untreated fecal waste accumulates everyday in tonnage quantities in municipal landfills and this represents a serious health hazard to the population. Human wastes are biodegradable in the landfill environment but their biodegradation is significantly impaired when they are wrapped or enclosed in non-biodegradable plastic films and fabrics.

In order to solve the above mentioned problems, it has been necessary to develop a degradable disposable diaper. The term degradable is used herein to denote that the materials of which the diaper is made will degrade within the landfill environment. In the case of cellulose or cellulose-based components, the degradation is primarily the result of the action of microorganisms (biodegradation). In the case of the components based on synthetic hydrocarbon thermoplastics, the degradation is a two-stage process, heat-induced abiotic oxidative degradation followed by the biodegradation of the oxidized molecular fragments (oxo-biodegradation).

The following patents describe some efforts to solve the above mentioned problems:

In U.S. Pat. No. 4,964,857 (Osborn) an improved diaper has first and second layers of absorbent material made of absorbent cotton and the first and second layers of moisture repellent material are made of a coated paper, normally coated with a natural wax. Both cotton and paper coated with natural wax are capable of complete biodegradation within a few weeks time. The coated paper, however, has some hardness and not too much flexibility, which causes the diaper to tear easily and to lack the required skin softness.

In U.S. Pat. No. 5,185,009 (Sitman) a biodegradable diaper comprises an outer sheet of biodegradable material able to resist water absorption; an inner sheet of biodegradable material able to allow the passage of water and attached to the outer sheet by a biodegradable adhesive at the periphery to form an envelope; a super absorbent core within the envelope; and a water resistant film of biodegradable material located within the core to assist in fluid distribution into the core. The outer sheet is of rayon, the inner sheet is of polyethylene, the adhesive is made of natural latex and the absorbent core is made of cellulose.

U.S. Pat. No. 5,542,940 (Jonker) describes a degradable disposable diaper that has an inner liner and an outer layer that are at least substantially made of a cellulosic material of the “ wet-strong long fiber” type. The web-strong long fiber paper serves to replace the synthetic non-woven inner liner and the polyethylene outer layer of the prior art disposable diaper.

In U.S. Pat. No. 5,759,569 (Hird) disposable diapers are described in which the inner and the outer sheet are made from trans-1,4-polyisoprene and similar trans-polymers. This application further relates to biodegradable polymers containing compositions comprising a blend of these transpolymers with other biodegradable components such as starch.

None of the disposable diapers in accordance with the above described U.S. patents have become a success, either because of the materials required, making the resultant product too expensive, or because they exhibited urine leakage, due to the layers not being strong enough in a wet condition, or because they are not comfortable to the wearer.

The above mentioned disadvantages have been overcome with the invention herein described.

### OBJECTS OF THE INVENTION

One object of the present invention is to provide a disposable diaper with a pervious topsheet and an impervious backsheet made from synthetic hydrocarbon thermoplastics having low cost, ease of fabrication and high wet strength.

Another object of the present invention is to provide a disposable diaper with a pervious topsheet and an impervious backsheet which are degradable after being used and discarded.

Still another object of the invention is to provide a disposable diaper with a degradable absorbent core.

Yet another object of the present invention is to provide a disposable diaper which is environmentally friendly.

A further object of the present invention is to provide a disposable diaper having enhanced properties with respect to absorption, comfort and strength, yet easy to produce and attractively priced.

Other objects of the invention will be pointed out herein after, or will be readily apparent to those skilled in the art, but it is to be understood that there are different embodiments within the scope of the invention and that the embodiments shown herein are used for illustrative purposes only.

### SUMMARY OF THE INVENTION

A degradable disposable diaper includes one or more sheets made of polyolefin with the polyolefin including a prodegradant causing the sheet to degrade. The prodegradant includes a metal compound such as a metal selected from the group consisting of cobalt, cerium, and iron. The preferred metal compound is a metal carboxylate. The polyolefin is preferably polyethylene or polypropylene. A secondary polyolefin may be used to aid the incorporation of the prodegradant into the primary polyolefin. A filler may also be used with the polyolefin and prodegradant. The filler preferably has a particle size less than 150 mesh and is free of water. The filler is preferably calcium carbonate having a 1 to 10 micron particle size. The sheet contains between about 0.001 and about 15 weight % prodegradant and most preferably between about 0.01 and about 3 weight % prodegradant. The sheet also includes up to about 15 weight % filler. The diaper also includes a degradable absorbent core.

The present invention is referred to as a degradable disposable diaper. The term ‘degradable’ as defined above represents a significant difference between the products of the present invention, which are degradable under landfill conditions, and those commercially available disposable diaper compositions that are not. Commodity thermoplastic films/fabrics in which high strength (including especially wet strength), ease of fabrication and reasonable cost are necessary are commonly made of polyolefins, in particular polyethylene and polypropylene. Although these thermoplastics have all the necessary physical and chemical properties required of the topsheet and backsheet components of disposable diapers, they persist for a very long time in, for example, a landfill environment. They are bioinert, i.e., they are not degraded by microorganisms, and they do not degrade significantly under the relatively benign conditions that exist in a typical landfill environment. They persist and accumulate and, moreover, they retard the biodegradation of biodegradable materials that they are wrapped around.

Plastics which will biodegrade in a landfill, such as linear polyesters or modified starch, are not suitable for incorporation in disposable diapers because they have one or more of the following undesirable characteristics: poor wet strength, poor physical and mechanical and properties, relatively high cost, incompatibility with existing fabrication equipment, excessive energy consumption associated with synthesis.

The degradable disposable diaper comprises an impervious backsheet that has incorporated in it a prodegradant which adds degradable properties. The backsheet surface is preferably positioned adjacent to the garment surface; a degradable pervious topsheet, also incorporating the prodegradant, is positioned adjacent the body surface and it is soft and comfortable to avoid any skin irritation; an absorbent core is placed between the topsheet and the backsheet; a pair of side edges are positioned adjacent to the absorbent core; an elastic zone positioned on the legs region on said side edges; elastic front and back waist regions; a pair of barrier cuffs disposed longitudinally between the front and back waist regions, and positioned over the absorbent core in order to have less space for the feces to flow over the topsheet, but instead to be absorbed. A fastening system is positioned in the front waist region over the backsheet external side, such system could be a pair of fastening tape-tabs with a pressure sensitive adhesive on one of its faces. The fact that both topsheet and backsheet are degradable helps the absorbent core and the feces to degrade at a time controllable rate which is much faster than it is for those disposable diapers that are available at present.

While the specification concludes with claims particularly pointing out and distinctly claiming the subject matter which is regarded as forming the present invention, it is believed that the invention will be better understood from the following detailed descriptions.

### BRIEF DESCRIPTION OF THE DRAWINGS

For a detailed description of the embodiment of the invention, reference will now be made to the accompanying drawings wherein:

FIG. 1 is a diagrammatic perspective view of a disposable diaper according to the present invention shown in the worn condition;

FIG. 2 is a diagrammatic perspective view of the disposable diaper of FIG. 1 shown in a laid out position with a portion thereof shown in a cut away view;

FIG. 3 is a cross-sectional view of a back sheet of the disposable diaper shown in FIGS. 1 and 2; and

FIG. 4 is a cross-sectional view of an alternative back sheet for the disposable diaper shown in FIGS. 1 and 2.

### DETAILED DESCRIPTION OF THE INVENTION

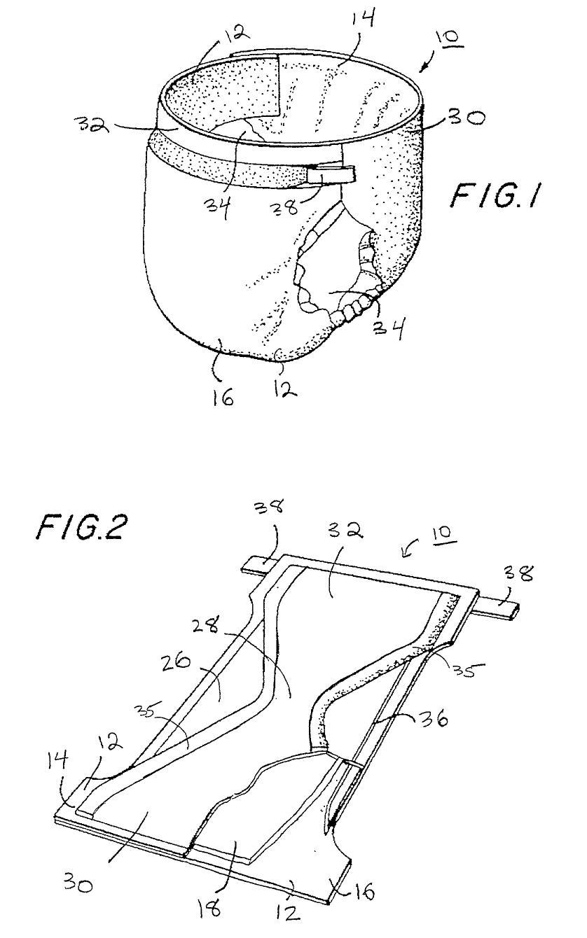

Referring initially to FIGS. 1 and 2, according to a preferred embodiment of the present invention, a disposable diaper 10 includes at least one degradable diaper sheet 12. Degradable diaper sheet 12 is made of a formulation which includes degradation properties and preferably includes a conventional polyolefin, such as polyethylene or polypropylene, referred to herein as the primary polyolefin, and a prodegradant imparting the degradable property. The prodegradant is adapted for incorporation into degradable diaper sheet 12 and contains ingredients that impart the degradable characteristics.

The prodegradant is preferably a metal compound. The metal compound includes a metal preferably selected from the group consisting of cobalt, cerium, and iron. It is understood that the metal may be in ionic form. Other suitable metals are aluminum, antimony, barium, bismuth, cadmium, chromium, copper, gallium, lanthanum, lead, lithium, magnesium, mercury, molybdenum, nickel, potassium, rare earths, silver, sodium, strontium, tin, tungsten, vanadium, yttrium, zinc or zirconium.

The metal compound may be in any suitable form for incorporation into a polyolefin material. In particular, the metal compound is preferably a metal carboxylate. For example, the metal carboxylate may be a metal stearate or a metal neodeconate. The metal is preferably selected from among the metals described above. Thus, exemplary preferred metal compounds include, but are not limited to, cobalt stearate, iron stearate, and cerium stearate. The metal compound is most preferably cobalt stearate.

Alternatively, the prodegradant could be substituted by any other prodegradant that produces the same degradability effect and characteristics.

A secondary polyolefin may be combined with the prodegradant prior to being mixed with the primary polyolefin to aid in the incorporation of the prodegradant into the primary polyolefin. Exemplary secondary polyolefins include low density polyethylene (LDPE), linear low density polyethylene (LLDPE), polypropylene, polybutylene and copolymers of ethylene such as polyethylene-vinyl acetate (EVA), polyethylene-acrylic acid (EAA), polyethylene-methacrylic acid EMA) or copolymers of ethylene or propylene with the lower olefins such as, butene-1, pentene-1,hexene or octene.

The term “polyethylene” as used herein includes any polymer or resin where ethylene is predominant and is illustrated by the polyethylene compounds in the foregoing list. Likewise, the term “polypropylene” as used herein includes any polymer or resin where polypropylene is predominant and is illustrated by the polypropylene compounds in the foregoing list. Further, it will be understood that the term “polyolefin” may include a mixture of polyolefins. Still further, it will be understood that herein, the term “polyolefin” may refer to the primary polyolefin, the secondary polyolefin, or a combination of both, as will be apparent from the context.

A filler may be added to reduce the amount of polyolefin required to produce sheet 12. The filler may be added to the primary polyolefin or can be combined with a mixture of secondary polyolefin and prodegradant. The filler is preferably selected from the inorganic carbonates synthetic carbonates, nepheline syenite, talc, magnesium hydroxide, aluminum trihydrate, diatomaceous earth, mica, natural or synthetic silicas and calcined clays or mixtures thereof, having a particle size less than 150 mesh. The filler is preferably free of water.

The inorganic carbonates such as calcium carbonate or magnesium carbonate are preferred as fillers; however, lithium carbonate, and sodium carbonate may also be used. In addition, the synthetic carbonates such as the hydrotalcite-like compound or the dihydroxyaluminum sodium carbonates may be used. In addition to the inorganic or synthetic carbonates, a filler such as nepheline syenite, talc, magnesium hydroxide, aluminum trihydrate, diatomaceous earth, mica, natural or synthetic silicas including silicon dioxide and calcined clays or mixtures thereof, having a particle size less than 150 mesh may be used. Fillers preferably have particle sizes less than 150 mesh but the smaller the particle size of the filler material, the more preferred it is as the filler material. The most preferred filler is calcium carbonate having a 1 to 10 micron particle size.

The preferred total amount of prodegradant is the one that imparts the degradability characteristics without affecting the diaper performance when used, while allowing a suitable shelf life.

Diaper sheet 12 preferably contains between about 0.001 and about 15 weight % prodegradant, more preferably between about 0.01 and about 3 weight % prodegradant. Diaper sheet 12 may optionally further include up to about 2 wt. % filler for a polypropylene based diaper sheet 12 and up to about 15 wt. % filler for a polyethylene based diaper sheet 12. The remainder is polyolefin. The total polyolefin includes any primary polyolefin and any second polyolefin, such as described above. Thus, diaper sheet 12 preferably contains from about 0.001 to about 15 wt % prodegradant, up to about 99.999 wt % polyolefin, and up to about 15 wt % filler, more preferably from about 0.01 to about 3 wt % prodegradant, from about 70 to about 99.99 wt % polyolefin, and up to about 15 wt % filler.

More particularly, a preferred formulation of diaper sheet 12 contains from about 0.01 to about 3 wt % of a metal carboxylate, preferably cobalt stearate, from about 70 to about 99.99 weight % of a polyolefin selected from the group consisting of polyethylene and polypropylene, and up to about 15 weight % of a filler.

Referring again to FIGS. 1 and 2, degradable diaper sheet 12 may be either of the exterior sheets of a diaper, i.e. topsheet 14 and the backsheet 16 conventionally termed the shell. Thus, disposable diaper 10 preferably includes the shell of topsheet 14, backsheet 16, and an absorbent core 18 disposed therebetween. Preferably each of topsheet 14, backsheet 16, and absorbent core 18 are degradable. It should be appreciated that any part of the diaper containing a polyolefin may be made degradable such as by adding a prodegradant to the polyolefin.

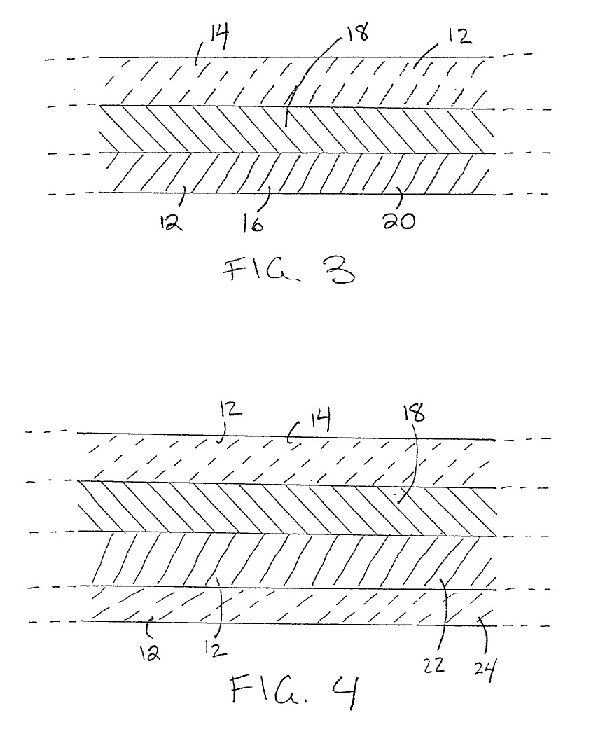

Referring to FIG. 3, backsheet 16 may include a single layer 20 of impervious polymeric film. Alternatively, referring to FIG. 4, backsheet 16 may include an impervious film layer 22 joined to a non-woven layer 24, such as by lamination. Topsheet 14 is typically formed of a non-woven fabric. An exemplary impervious polymeric film is used on polyethylene, whereas an exemplary non-woven fabric is based on polypropylene.

It will be understood that disposable diaper 10 may optionally include other conventional diaper components. A diaper component, containing a polyolefin, preferably also contains a prodegradant such as described above, preferably in an amount to render the component degradable.

It will be understood that a degradable diaper sheet according to a preferred embodiment of the present invention may be any conventional diaper layer made from a polyolefin. Exemplary diaper layers include, but are limited to, an interior sheet between a topsheet and a backsheet, a wicking layer between a topsheet and an interior sheet, and the like.

Referring again to FIGS. 1 and 2, according to an exemplary embodiment, disposable diaper 10 includes a pair of longitudinal side edges 26, a crotch region 28, a front waist region 30, a back waist region 32, topsheet 14, preferably permeable such that it allows liquids to penetrate through its thickness, backsheet 16, preferably impervious, a degradable absorbent core 18 positioned in between the topsheet and the backsheet, a pair of barrier cuffs 35, a pair of elastically contractible gasketing cuffs 36, and a pair of fastening members 38 positioned in the front waist region over the backsheet external side. An exemplary fastener system is of the type including a pair of fastening tape-tabs with a pressure sensitive adhesive on one of its faces, and a pair of elastic zones positioned on the waist regions. The waist regions may each comprise a foam comfort waistband. Fastening members 38 are preferably degradable fastening members, more preferably incorporating the polyolefin materials containing prodegradant as herein described. Further, a comfort waistaband is preferably a degradable waistband, more preferably incorporating the polyolefins materials containing prodegradant as herein described. Still further, it is contemplated that any other of the diaper components that incorporate polyolefin preferably also incorporate prodegradant such that the diaper component is degradable.

The diaper is preferably made with elastic and joining means like hotmelt adhesives or any other means known in the state of art. The longitudinal side edges together with the waist regions and the crotch zone make up the diaper periphery.

The topsheet is compliant, soft feeling, and non-irritating to the wearer's skin. Further, the topsheet is liquid pervious allowing liquids to readily penetrate through its thickness to the absorbent core. The topsheet functions include to maintain the wearer's body isolated from the absorbent core material and to avoid the absorbent core wetness at the same time that it is the body exudates recipient. It has been found that when the topsheet incorporates the prodegradant, it gives to the diaper degradable characteristics when added in a range preferably between 0.001 and 15wt %, more preferably 0.01 and 3 wt %, without damaging its softness and tear strength characteristics during its wearing. The prodegradant component in the topsheet promotes the oxidative degradation of the polymer molecules, as evidenced by a reduction in mechanical properties such as tear strength and elongation at break. The degradation can be initiated by ultraviolet (UV) light or by heat even at the moderate temperatures found in a landfill or the higher temperatures that occur in composting operations. The prodegradant component greatly accelerates the degradation which has been initiated in these ways. This degradation is noticed with >50% loss of physical and mechanical properties such as physical embrittlement and disintegration as well as decrease in tear and elongation properties. The intermediate and ultimate degradation products are identical to those that would form in the absence of the prodegradant formulations but they form one or two orders of magnitude faster with the formulations. The prodegradant-containing material herein mentioned may be incorporated in the topsheet as a result of being mixed in at the time the polyolefin pellets are fed into the extruder.

The topsheet size depends on the absorbent core size and the whole diaper design. As an example the topsheet length could be between 32 and 55 cm, and between 11 and 19 cm wide.

A particularly preferred topsheet comprises staple length non-woven polypropylene fibers. Some of the physical characteristics of this material are a base weight around 14 to 19 g/m², with a tear strength in the machine longitudinal direction between 1600 and 1900 g/in, and in the transverse direction between 900 and 1300 g/in. It has an elongation in the machine direction between 40 and 60%, and transversely between 50 and 60%. This topsheet has to have a surfactant treatment in order to be hydrophilic to permit liquids to readily penetrate through its thickness. The surfactant treatment could be done totally or partially depending on the design requirements. The topsheet could carry other additives for the wearer's skin health, for example some oil cremes or natural products like Aloe Vera.

The backsheet is liquid impervious and is preferably manufactured from a thin and flexible plastic film. The backsheet prevents the exudates absorbed and contained in the absorbent core from wetting articles which contact the diaper.

The backsheet is positioned adjacent to the absorbent core back surface and it is in contact with the wearer's garment. The backsheet is joined by any attachment means known in the art, for example, a uniform continuous layer of hot melt adhesive, a patterned layer of adhesive (spirals, continuous lines, dots, etc.)

The backsheet could be manufactured from any flexible plastic material which is liquid impervious, and that works as an exudates container. Preferably, the backsheet is a polyethylene film having a base weight between 20 and 26 g/m² with a tear resistance between 1300 and 2000 g in the machine direction and between 1050 to 1700 g on the transverse direction. Other materials that could be used in the backsheet manufacture include various polyethylene or polypropylene films, woven or non-woven fabrics, co-polymers, ter-polymers, and other thermoplastic materials.

Further, the backsheet may permit vapors to escape from the absorbent core while still preventing exudates from passing through the backsheet; it may have breathable characteristics. It could be made from a laminated material which has the appearance of a conventional textile. In order to acquire its degradable characteristics, the backsheet has the same prodegradant treatment as the topsheet. The prodegradant is preferably incorporated at a level of preferably between 0.001 and 15 wt %, more preferably 0.01 and 3 wt %

The size of the backsheet is dictated by the diaper design. The backsheet is extended to form the longitudinal diaper edges and the waist and crotch regions, that altogether comprise the diaper periphery.

The absorbent core is positioned between the backsheet and the topsheet. It is generally manufactured by any means which allows it to be compressible, conformable, non-irritating to the wearer's skin, capable of absorbing and retaining fluids and certain body exudates. The absorbent core may be manufactured in a wide variety of sizes and shapes (e.g., rectangular, hourglass, etc) and from a wide variety of liquid absorbing materials commonly used in disposable diapers and other absorbent articles, such as comminuted wood pulp which is generally referred to as airflet. Examples of other suitable absorbent materials include creped cellulose wadding, absorbent foams, absorbent sponges, super absorbent polymers, or any similar material or combination of materials known in the art. Therefore, the dimensions, shape and configuration of the absorbent layer may be varied (e.g., the absorbent core may have a varying thickness or a hydrophilic gradient design, superabsorbent gradient, low density zones, etc.). Also the total absorbent capacity depending on the wearer's sizes may vary too.

A preferred absorbent core design comprises a homogeneous mixture of cellulose natural fibers with superabsorbent polymer (SAP) granules made of sodium polyacrylate. The main functions of the SAP component are the liquid absorption and retention in order to avoid any leakage. The mixture of SAP/cellulose may vary in component proportions. Compression of the mixture is needed to get the desired density.

The absorbent core may have another polypropylene fiber liquid distribution layer which allow the liquids to move to the different absorbent core regions. This polypropylene may also be rendered degradable by the incorporation of the prodegradant.

The elastically contractible gasketing cuff is formed from the backsheet extension on the crotch region and it comprise several elastic components. The gasketing cuff function is to draw and hold the diaper against the legs of the wearer in order to provide a seal which prevents any leakage. The elastic components are secured to the cuff in an elastically contractible condition so that in a normally unrestrained configuration, the elastic material effectively contracts the cuff material adjacent to it around the legs of the wearer. The elastic material can be affixed to the cuff by any of the state of the art methods. The gasketing cuff is made from an impervious material, therefore the liquids can not leak.

Degradable barrier cuffs may be manufactured from a wide variety of materials, e.g., elastomeric films, non-woven polypropylene, or laminated materials. The characteristic degradability is obtained by means of prodegradant incorporation at a level preferably between 0.001 and 15 wt %, more preferably 0.01 and 3 wt %, a level at which there will be no deleterious effects during use. A preferred material used for the barrier cuffs is hydrophobic non-woven polypropylene.

The barrier cuffs are preferably disposed longitudinally over the disposable diaper. The preferred embodiment is to dispose the barrier cuffs over the absorbent core, therefore a reception channel of approximately 10 cm for the exudates is formed. This reception channel allows the wearer exudates to stay for a longer time in contact with the absorbent core. Therefore, the absorbent core has a better performance in the way of avoiding leakage. It will be understood that the position of the barrier cuffs may vary according to the design of a diaper.

Degradable fastening members may be manufactured from a wide variety of materials, e.g., elastomeric films, non-woven polypropylene, or laminated materials. The characteristic degradability is obtained by means of prodegradant incorporation at a level preferably between 0.001 and 15 wt %, more preferably 0.01 and 3 wt %, a level at which there will be no deleterious effects during use. A preferred material used for the fastening members is hydrophobic non-woven polypropylene.

The prodegradant is preferably incorporated into a diaper component by any process which is conventionally used to produce a diaper component. For example, the prodegradant may be provided in pellet form, suitable for combination with pellets of conventional diaper component material, such as polypropylene or polyethylene in a film-making process. Thus, according to a preferred embodiment of the present invention, a method of making a degradable diaper includes providing a degradable polyolefin melt blown film, preferably a chemically degradable polyolefin melt blown film.

The term “degradable” as used to describe the polyolefin film means that the polyolefin film exhibits more than loss of physical properties such as brittleness or lost of tensile strength but loss of molecular weight as well. The degradation of the polyolefin resins is clearly characterized as chemical since the degradation does not require UV light (photodegradable) or a bio initiator (biodegradable) for the degradation to be initiated and to take place. On the other hand, UV light (sunlight) will accelerate chemical degradation.

Melt blowing of polyolefins is a well known and established process for making polyolefin film. In the melt blowing process, the polyolefin is extruded through a circular die having a gas, usually air, blown into the circle of resin to form a bubble which is pulled upward until the resin cools and the resin film is then run through rollers which collapse the bubble and form a continuous cylinder of film. The apparatus for melt blowing film consists of an extruder, a circular die, an air ring for blowing air into the die, a collapsible frame for collapsing the bubble formed, nip rolls and a winder for the collapsed film. The film is then slit to form a sheet which usually is twice the size of the collapsed film (a cylinder of film) or in other words is about the size of the circumference of the bubble formed.

According to the present invention, melt blown polyolefin films are chemically degradable when a certain chemical prodegradant, preferably a metal carboxylate, is introduced to the polyolefin. The prodegradant may be introduced as part of a prodegradant containing material having a secondary polyolefin and/or filler. The degradable resins of the present invention are produced by incorporating the combination of certain optional fillers, which may be mixtures of fillers, and the chemical prodegradant, a metal carboxylate, to resins which are able to be melt blown into film and mixing the filler and prodegradant in an extruder and then forming the pellets which are used in the conventional melt blown film equipment.

The surface of the filler is preferably treated so as to not adsorb water which will produce steam and holes when melt blown into film. Accordingly, these fillers, such as calcium carbonate, are treated with organic acids to assist the processability of the carbonate and produce a more hydrophobic filler product. Acids such as stearic or oleic acid are conventional acids for surface treating the carbonates or other fillers. The surface treatment is usually done by the carbonate supplier.

Polyolefin resins that are injection molding resins or are unsuitable for the preferred melt blowing process are characterized as having small ultimate elongations, usually 100 to 300%, with no melt strength.

The absorbent articles manufactured according to the present invention are environmentally friendly diapers because the components are degradable after use.

### EXAMPLE 1

Diapers were used in which the polyethylene film contained 98.5 wt % polyethylene, 1.44 wt % calcium carbonate, and 0.06 wt. % cobalt stearate and the polypropylene non-woven fabric contained 98.5 wt. % polypropylene resin, 0.75 wt % polyethylene resin, 0.7275 wt. % calcium carbonate, and 0.0225 wt. % cobalt stearate.

Tensile breaking strength, elongation at break, and tear strength measurements were made on complete diapers over the tape tabs zones. Measurements were stopped when fragmentation of the film or the non-woven was observed. Results are shown in Table 1. The test temperature was 60°C.

TABLE 1

| Time (hrs) | Peak force (Kg) | Elongation (%) | Tear strength (Kg) |
|---|---|---|---|
| 0 | 5.48 | 37.45 | 4.77 |
| 24 | 5.04 | 30.63 | 4.26 |
| 32 | 4.20 | 31.21 | 4.55 |
| 40 | 4.67 | 29.07 | 4.20 |
| 48 | 6.65 | 31.70 | 5.43 |
| 64 | 5.82 | 39.48 | 5.17 |
| 72 | 4.61 | 27.8 | 4.15 |
| 80 | 4.79 | 26.23 | 4.2 |
| 88 | 4.55 | 26.56 | 4.02 |

Reductions in tensile properties were observed in the range of 32 to 40 hours and again at 72 hours and beyond, owing to the action of the prodegradant. An augmented tear strength is observed at 48 hours, but this property also decreases significantly after 72 hours, owing to the action of the prodegradant. What is illustrated here is a period during which the integrity and serviceability of the diaper is maintained, followed by a steady loss of mechanical strength as a result of the incorporation of prodegradant in the polyolefin components.

### EXAMPLE 2

Fragmentation test

The time required for a non-woven polypropylene fabric to become brittle (break into fragments) was measured with and without prodegradant at different oven temperatures. The non-woven polypropylene fabric contained 98.5 wt. % polypropylene resin, 0.75 wt % polyethylene resin, 0.7275 wt. % calcium carbonate, and 0.0225 wt. % cobalt stearate.

These data are shown in Table 2. NF means no fragmentation.

TABLE 2

| % Additive | 40° C. | 50° C. | 60° C. | 70° C. |
|---|---|---|---|---|
| 0 | NF | NF | NF | NF |
| 1.5 | NF | 3 weeks | 2 weeks | 1 week |

The increase in degradability in the presence of the additive as a function of increasing temperature is obvious.

### EXAMPLE 3

Times required for fragmentation at different temperatures, using complete diapers in the tests, are shown in Table 3; the higher the temperature, the shorter the time required for embrittlement. The diapers included a polyethylene film that contained 98.5 wt % polyethylene, 1.44 wt % calcium carbonate, and 0.06 wt. % cobalt stearate and a polypropylene non-woven fabric that contained 98.5 wt. % polypropylene resin, 0.75 wt % polyethylene resin, 0.7275 wt. % calcium carbonate, and 0.0225 wt. % cobalt stearate.

TABLE 3

| Time | Temperature |
|---|---|
| 3 days | 70° C. |
| 1 week | 60° C. |
| 22 days | 43° C. |

### EXAMPLE 4

This heatage study illustrates the shelf life and the selection of the appropriate type of packaging. The samples used were complete diapers in standard packaging. The diapers incorporated a polyethylene (PE) film that contained 98.5 wt % polyethylene, 1.44 wt % calcium carbonate, and 0.06 wt. % cobalt stearate and a non-woven polypropylene (PP) that contained 98.5 wt. % polypropylene resin, 0.75 wt % polyethylene resin, 0.7275 wt. % calcium carbonate, and 0.0225 wt. % cobalt stearate. One whole un-opened package of diapers was placed in heatage ovens with temperatures of 43, 54 and 71° C. At approximately three weeks (actually 22 days) the packages were removed and inspected. The following are the observations made during the opening and inspection of the packages and diapers. The testing followed ASTM method D 5510.

After heating at 43° C., the packaging appeared to be un-affected, and remained intact and sealed. The package was opened at the top as any consumer normally would open it. The diapers looked normal in color and no signs of degradation were noticed. A diaper was removed and inspected closely. It did not break, tear or come apart upon applying a strong pull. The diaper appeared to be un-changed.

After heating at 54° C., the packaging appeared to be un-affected, and remained intact and sealed. The package was opened at the top as any consumer normally would open it. A distinct change in the color of the foam comfort strip was immediately noticed. In particular, the color had yellowed somewhat as compared to the 43° C. treated samples. Visually, there were no signs of fragmentation of either the PP or PE materials. A diaper was removed and its brittle texture was noted. Both the PP and PE materials were brittle and could be torn with little or no effort. (Photo #2)

After heating at 71° C., the packaging appeared to be un-effected, and remained intact and sealed. The package was opened at the top as any consumer normally would open it. A profound change in the color of the foam comfort strip was immediately noticed. In particular, it had yellowed more strongly than the 54° C. treated sample, and the PE and PP materials were clearly fragmented to a severe degree. Upon removal of a diaper, the diaper was in a very fragile condition, the PE film was fragmented and pieces fell off the diaper. The diaper was then unfolded, and the diaper fell apart. The only parts that remained intact were the two tapes and the decorative front strip on which the tapes would normally be applied.

### EXAMPLE 5

This heatage study illustrates the shelf life of the degradable diaper and the selection of the appropriate type of packaging. The samples used were complete diapers in green commercial packaging. The diapers incorporated a polyethylene (PE) film that contained 98.5 wt % polyethylene, 1.44 wt % calcium carbonate, and 0.06 wt. % cobalt stearate and a non-woven polypropylene (PP) that contained 98.5 wt. % polypropylene resin, 1.2975 wt % polyethylene resin, 0.045 wt % zinc oxide, 0.045 wt % titanium dioxide, and 0.1125 wt. % cesium stearate. A single whole unopened package of diapers was placed in heatage ovens with temperatures of 43, 54 and 71° C. Since this packaging was opaque, photos were only taken at 30 days. At thirty days, the packages were removed and inspected. The following are the observations made during the opening and inspection of the diapers. The testing followed ASTM method D 5510.

For reference, the heatage samples were compared to unopened control sample packages stored in the laboratory at 23° C.

After heating at 43° C., No color change or odor was noticed. No obvious signs of degradation/fragmentation of either PP or PE materials. One diaper was torn apart by hand and was as strong as the Control Sample.

After heating at 54° C., only a very slight yellow color was noticed, and no detectable odor. Most of the color change was noticed in the comfort strips and around the hot melt adhesive areas. No obvious signs of degradation/fragmentation of either PP or PE materials were observed. One diaper was torn apart by hand and was as strong as the Control Sample and 43° C.

After heating at 71° C., a very noticeable yellow color was seen in the PP material. It was even more noticeable in the front and rear foam comfort strips and the hot melt adhesive throughout the diaper; an obvious odor was also noticed immediately. The diaper was fragmented at the outside hook and loop and leg cuff areas. The PP material, inside and out was fragmenting and flaking away, but areas with hot melt seemed to hold together better than areas without. Areas with multiple layers, i.e. PP, PE and tissue although brittle and weak were mostly intact.

The testing using 43 & 54° C. heating illustrates how the diapers will remain intact even during adverse storage conditions. The testing using 71° C. heating clearly illustrates the degradability of the product. It is believed, from this testing, that the diapers are characterized by at least a six months shelf life, including when subjected to adverse conditions such as two weeks of extreme storage temperatures not to exceed ˜54° C.

### EXAMPLE 6

This heatage study illustrates the shelf life and the selection of the appropriate type of packaging. The samples used were complete diapers in clear packaging. The diapers incorporated a polyethylene (PE) film that contained 98.5 wt % polyethylene, 1.44 wt % calcium carbonate, and 0.06 wt. % cobalt stearate and a non-woven polypropylene (PP) that contained 98.5 wt. % polypropylene resin, 0.75 wt % polyethylene resin, 0.7275 wt. % calcium carbonate, and 0.0225 wt. % cobalt stearate. Two whole unopened packages of diapers were placed in heatage ovens with temperatures of 43, 54 and 71° C. Photos were taken at 11, 22 and 30 days. At thirty days, one package from each oven was removed and inspected. The following are the observations made during the opening and inspection of the diapers. The testing followed ASTM method D 5510.

For reference, the heatage samples were compared to unopened control sample packages stored in the laboratory at 23° C.

After heating at 43° C., no color change or odor was noticed. No obvious signs of degradation/fragmentation of either PP or PE materials. One diaper was torn apart by hand and was as strong as a Control Sample.

After heating at 54° C., only a very slight yellow color was noticed, but no odor. No obvious signs of degradation/fragmentation of either PP or PE materials. One diaper was torn apart by hand and was as strong as the Control Sample and 43° C.

After heating at 71° C., a very noticeable yellow color was seen in the PP material and even more noticeable the front and rear foam comfort strips and the hot melt adhesive throughout the diaper. An obvious odor was also noticed immediately. With the diaper held up to the light, as were the other samples to inspect the PE material, obvious degradation/fragmentation of the PE material was noticed. Upon tearing the diaper open, the PP material was slightly weak as compared to the other samples, and the fragmentation of the PE material was quite extensive.

The PE material was a complete success since it lasted for 30 days at 54° C., and completely fragmented at 71° C.

### EXAMPLE 7

A polyethylene cast film containing 98.5 wt % polyethylene, 1.35 wt % calcium carbonate, and 0.15 wt. % cobalt stearate was studied. Testing was carried out to determine certain properties listed in Table 4, according to specific methods. The results are listed in Table 4. The exposure was ambient outdoor exposure.

TABLE 4

| | | ACTUAL TEST | |
|---|---|---|---|
| TEST | METHOD | VALUES | |
| Original Melt Index | ASTM D 1238 (190/2.16) | 3.94 | g/10 min. |
| Exposed Melt Index | ASTM D 1238 (190/2.16) | 37.02 | g/10 min. |

### EXAMPLE 8

Complete diapers that incorporated a polyethylene cast film containing 98.5 wt % polyethylene, 1.35 wt % calcium carbonate, and 0.15 wt. % cobalt stearate were studied. Testing was carried out to determine certain properties listed in Table 5, according to specific methods listed in Table 5. The results are listed in Table 5.

TABLE 5

| | | ACTUAL TEST |
|---|---|---|
| TEST | METHOD | VALUES |
| Heatage | ASTM D 5510 | 160° F./71° C. |
| Thermal Degradation | (Days until fragmentation) | 6 |
| Ambient | ASTM D 5272 | Days |
| Outdoor Exposure | (Days until fragmentation) | 35 |
| QUV | ASTM D 5208 | Hrs. |
| Accelerated Weathering | (Hours until fragmentation) | 72 |

### EXAMPLE 9

Complete diapers that incorporated a polyethylene cast film containing 98.5 wt % polyethylene, 1.35 wt % calcium carbonate, and 0.15 wt. % cobalt stearate were studied.

QUV accelerated weathering tests were performed according to ASTM method D 5208, with the results listed in Table 6, for various exposure times. In Table 6, MD indicates machine direction.

TABLE 6

| Property | 0 hrs. | 24 hrs. | 48 hrs. | 72 hrs. | 96 hrs. | 168 hrs. |
|---|---|---|---|---|---|---|
| Tensile (psi) MD | 1,355 | 1,950 | 1,517 | 821 | 837 | 1,346 |
| Elongation (%) | 483 | 523 | 400 | 272 | 198 | 6 |
| MD | | | | | | |

### EXAMPLE 10

Testing was carried out for four different non-woven polypropylene fabric compositions containing and for complete diapers incorporating the same four a non-woven polypropylene fabric compositions. Each composition contained primarly polyolefin, with small amounts of prodegradant. In particular, the amount of cobalt stearate contained in Compositions 1, 2, 3, and 4 was 0.0225 wt. %, 0.033 wt. %, 0.045 wt. %, 0.075 wt. %, respectively. A control fabric and diapers containing the control fabric were also test. The control fabric had a composition of about 100% polypropylene and about 0% prodegradant.

QUW tests were carried out to determine the properties listed in Table 7, according to the methods listed in Table 7, with the results listed in Table 7. In Table 7, MD indicates machine direction.

TABLE 7

| Accelerated Weathering | 0 hrs. | 24 hrs. | 48 hrs | 72 hrs. | 96 hrs. |
|---|---|---|---|---|---|
| Control | | | | | |
| ASTM D 5208 | | | | | |
| Tensile (psi) MD | 965 | 798 | 1,055 | 773 | 1,369 |
| Elongation (%) MD | 41 | 41 | 43 | 42 | 39 |
| Composition 1 | | | | | |
| ASTM D 5208 | | | | | |
| Tensile (psi) MD | 392 | 507 | 213 | 346 | 689 |
| Elongation (%) MD | 43 | 39 | 27 | 36 | 17 |
| Composition 2 | | | | | |
| ASTM D 5208 | | | | | |
| Tensile (psi) MD | 504 | 460 | 597 | 283 | 101 |
| Elongation (%) MD | 37 | 36 | 35 | 19 | 15 |
| Composition 3 | | | | | |
| ASTM D 5208 | | | | | |
| Tensile (psi) MD | 425 | 636 | 522 | 544 | 316 |
| Elongation (%) MD | 43 | 39 | 35 | 30 | 19 |
| Composition 4 | | | | | |
| ASTM D 5208 | | | | | |
| Tensile (psi) MD | 587 | 738 | 331 | 173 | 46 |
| Elongation (%) MD | 33 | 31 | 24 | 2 | 3 |

While particular embodiments of the present invention have been illustrated and described, it would be obvious to those skill in the art that various other changes and modifications can be made without departing from the spirit and scope of the invention. It is therefore intended to cover in the appended claims all such changes and modifications that are within the scope of this invention.

1. A diaper comprising:
at least one sheet made of polyolefin, said polyolefin including a prodegradant causing the sheet to degrade.

2. The diaper of claim 1 wherein said prodegradant is a metal compound.

3. The diaper of claim 2 wherein said metal compound includes a metal selected from the group consisting of cobalt, cerium, and iron.

4. The diaper of claim 2 wherein said metal compound is a metal carboxylate.

5. The diaper of claim 1 wherein said polyolefin is selected from the group consisting of polyethylene and polypropylene.

6. The diaper of claim 1 wherein said polyolefin is a primary polyolefin and further including a secondary polyolefin selected to aid incorporation of the prodegradant into said primary polyolefin.

7. The diaper of claim 7 wherein said secondary polyolefins include low density polyethylene, linear low density polyethylene, polypropylene, polybutylene and copolymers of ethylene.

8. The diaper of claim 1 further including a filler with the polyolefin and prodegradant.

9. The diaper of claim 8 wherein said filler is selected from the group consisting of inorganic carbonate, synthetic carbonates, nepheline syenite, talc, magnesium hydroxide, aluminum trihydrate, diatomaceous earth, mica, silicas, and calcined clays.

10. The diaper of claim 8 wherein said filler has a particle size less than 150 mesh.

11. The diaper of claim 8 wherein said filler is free of water.

12. The diaper of claim 8 wherein said filler is calcium carbonate having a 1 to 10 micron particle size.

13. The diaper of claim 1 wherein said sheet contains between about 0.001 and about 15 weight % prodegradant.

14. The diaper of claim 1 wherein said sheet contains between about 0.01 and about 3 weight % prodegradant.

15. The diaper of claim 1 wherein said sheet includes up to about 15 weight % filler.

16. The diaper of claim 1 wherein said sheet contains from about 0.01 to about 3 weight % of a metal carboxylate.

17. A diaper, comprising:
a diaper having components made of polyolefins, all said polyolefins being degradable.

18. The diaper of claim 17 wherein all said polyolefins include a prodegradant.

19. The diaper of claim 17 wherein said diaper includes a degradable absorbent core.

20. The diaper of claim 17 wherein said polyolefins are chemically degradable.

21. A degradable disposable diaper which comprises: a degradable, pervious topsheet for exudates reception; a degradable, impervious backsheet; a degradable, absorbent core placed between said topsheet and said backsheet; a pair of side edges adjacent to the absorbent core; an elastic zone positioned on the legs region on said side edges; an elastic front and back waist region;; a fastening member positioned in the front waist region over the external side of the backsheet, where at least said topsheet and said backsheet are manufactured so as to incorporate a prodegradant that will give them degradability characteristics.

22. The diaper of claim 21 wherein said topsheet is manufactured using a non-woven material which incorporates a prodegradant in the range 0.01 to 3% by weight to provide degradability.

23. The diaper of claim 21 wherein said backsheet incorporates a prodegradant in the range 0.01-3% by weight to provide degradability.

24. The diaper of claim 21 wherein said diaper further comprises a pair of barrier cuffs disposed longitudinally between the front and back waist region, and positioned over the absorbent core, in order to leave less space for the feces to flow over the topsheet but instead to be absorbed, wherein said barrier cuffs incorporate a prodegradant in the range 0.01-3% by weight to provide degradability.

25. The diaper of claim 21 wherein said absorbent core is manufactured with cellulose or cellulose derivatives which are biodegradable or with a synthetic polymer that is also biodegradable, or some mixture or bled thereof.

26. The diaper of claim 21 wherein said fastening member incorporates a prodegradant.

27. A degradable disposable diaper which comprises: a degradable, pervious topsheet for exudates reception; a degradable impervious backsheet; a degradable absorbent core placed between said topsheet and said backsheet; a pair of side edges adjacent to the absorbent core; an elastic zone positioned on the legs region on said side edges; an elastic front and back waist region, and a fastening member positioned in the front waist region over the external side of the backsheet where at least said topsheet and said backsheet are manufactured with a non-woven material which incorporates a prodegradant that will give them degradability characteristics.

28. The diaper of claim 27 wherein said backsheet incorporates a prodegradant in the range 0.01-3% by weight to provide degradability.

29. The diaper of claim 27 wherein said topsheet incorporates a prodegradant in the range 0.01-3% by weight to provide degradability.

30. The diaper of claim 27 wherein said diaper further comprises a pair of barrier cuffs disposed longitudinally between the front and back waist region, and positioned over the absorbent core, in order to leave less space for the feces to flow over the topsheet but instead to be absorbed, wherein said barrier cuffs incorporate a prodegradant in the range 0.01-3% by weight to provide degradability.

31. The diaper of claim 27 wherein said absorbent core is manufactured with cellulose or cellulose derivatives and is biodegradable, or with a synthetic polymer that is also biodegradable, or some mixture or blend thereof.

32. The diaper of claim 27 wherein said fastening member incorporates a prodegradant.
